(19) United States
(12) Patent Application Publication
(10) Pub. No.: US 2005/0232868 A1
(43) Pub. Date: Oct. 20, 2005

(54) Methods of entrapping, inactivating, and removing viral infections by the administration of respiratory tract compositions

(75) Inventors: Paul John Rennie, Godalming Surrey (GB); Jayant Ekanth Khanolkar, Surbiton Surrey (GB); George William Jessen, Camberley (GB)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/979,498

(22) Filed: Nov. 2, 2004

Related U.S. Application Data
(63) Continuation-in-part of application No. 09/692,634, filed on Oct. 19, 2000.
Application No. 09/692,634 is a continuation-in-part of application No. 09/421,131, filed on Oct. 19, 1999, now abandoned.

(51) Int. Cl. A61L 9/04; A61K 31/315; A61K 35/78

(52) U.S. Cl. 424/45; 424/754; 424/755; 424/760; 514/494

(57) ABSTRACT

The present invention is directed to methods of preventing and treating respiratory tract viral infections by administering compositions to areas of the respiratory tract such as the nasal cavity, wherein the compositions provide for the encapsulation, inactivation, and/or removal of viruses and/or viral strains associated with the common cold and influenza. The methods of encapsulation, inactivation, and removal of cold and influenza viruses have been shown to create and maintain environments that are hostile to the viruses to result in effective prevention and treatment of cold and influenza-like symptoms.

### CROSS REFERENCE TO RELATED APPLICATION

This is a continuation-in-part of application Ser. No. 09/692,634 (P&G Case 8308), filed on Oct. 19, 2000, which is a continuation-in-part of application Ser. No. 09/421,131 (P&G Case 7831), filed on Oct. 19, 1999.

### FIELD OF THE INVENTION

The present invention is directed to methods of entrapping, inactivating, and/or removing viral infections by the administration of respiratory tract compositions. In particular, the present invention is directed to methods of entrapping, inactivating, and/or removing upper respiratory tract viral infections by the administration of respiratory tract compositions to the nasal cavity.

### BACKGROUND OF THE INVENTION

It is known that many different viruses and viral strains bring on symptoms associated with respiratory viral infections. The common cold is a complex syndrome caused by over 200 antigenically different viruses found in five virus families. These families include rhinovirus, myxovirus, paramyxovirus, respiratory syncytial virus, adenovirus and coronavirus. The most important group is rhinovirus, Gwaltney J. M., Common Cold, pp 489-493, Mandell G. L., Douglas, R. G. Jr., Bennett, J. E., Principles and Practice of Infectious Diseases, 3^{rd} ed., Churchill Livingstone, New York, 1990. Pinpointing the specific cause of the illness is difficult and not practical since there are also a number of predisposing factors whose contribution to the manifestation of symptoms is not fully understood. Such include, but are not limited to, physical fatigue, psychological stress, and overall physical healthiness.

Regardless of the virus and associated factors leading to the onset of cold and influenza symptoms, a number of remedies to alleviate the symptoms of the common cold and influenza have been suggested. In an attempt to improve existing cold remedies, experts in the field have suggested several alternative pharmacotherapies and subsequently conducted cold trials to test their efficacy, see for example the therapy disclosed in The New England Journal of Medicine published in 1986 and the therapy disclosed in The Journal of Infectious Diseases published in 1992. Treatment for influenza includes vaccination and use of specific antiviral drugs such as those treatments reviewed by A. Elliot and J. Ellis, 2000, Pharmaceutical Journal, 265, 446-451.

A number of patents have also been issued disclosing compositions for prevention and treatment of the common cold and/or influenza, and their methods of use. For example, U.S. Pat. Nos. 5,240,694, 5,422,097, and 5,492,689, all to Gwaltney, disclose treatment using combinations of anti-viral and anti-inflammatory compounds; U.S. Pat. Nos. Re 33,465 and 5,409,905, both to Eby disclose treatment using zinc salts; U.S. Pat. No. 5,626,831 to Van Moerkerken discloses treatments using orally administered aminocarboxylic acid compounds; U.S. Pat. Nos. 4,619,934 and 4,552,899, both to Sunshine, disclose treatment of cough and colds using compositions comprising non-steroidal anti-inflammatory drugs such as NSAIDS with antihistaminically effective materials such as chlorpheniramine; and EP 310317 to Bordt et al., assigned to Beecham, discloses a method for inactivating viruses and bacteria (e.g. vaccines) with pharmaceutical compositions wherein the method involves the inactivation of viruses or bacteria with ascorbic acid or its salts in the presence of oxygen and heavy metal ions.

Other disclosures of compositions, and their methods of use, include those publications which describe the administration of pharmaceutical compositions to the nasal membrane. For example, U.S. Pat. No. 4,689,223, issued Aug. 25, 1987, assigned to T&R Chemicals, discloses nasal spray compositions for treating the symptoms of or preventing the common cold, wherein the compositions comprise sulphites or bisulphites having low, but, no specific pH is disclosed. U.S. Pat. No. 6,080,783, issued Jun. 27, 2000, assigned to Gum Tech International, Inc., discloses viscous gels for delivering minor effective homeopathic amount of zinc or another metal to the nasal membrane. U.S. Pat. No. 4,767,788 to Diana, assigned to Sterling Drug Inc., discloses processes for destroying viruses such as rhinovirus with glutaric acid in the nasal mucosa. U.S. Pat. No. 5,622,724 to Bryce-Smith discloses spray preparations such as nasal sprays for treating symptoms of the common cold wherein the preparations comprise unchelated zinc compounds.

Although it is well documented that there exist numerous cough/cold products and remedies that are suitable for treating and/or preventing symptoms related to the common cold and influenza, it has not been discussed or found that a more effective method of treating cold and influenza symptoms includes the encapsulation, inactivation, and removal of respiratory tract viruses and viral strains. It has been found that at the onset of cold and influenza symptoms, these symptoms can be effectively alleviated through the use of methodologies involving encapsulation, inactivation, and/or removal of the viruses and/or viral strains. These methodologies have been shown to not only effectively treat cold and influenza symptoms, but such methods are also effective in treating and/or preventing reoccurrence of cold and influenza symptoms.

### SUMMARY OF THE INVENTION

The present invention is directed to methods of preventing and treating upper respiratory tract viral infections by administering a composition to the nasal cavity, wherein the composition comprises combinations of encapsulation, inactivation, and secretion or removal agents, such combinations being selected from (A) a rheological agent providing for a composition viscosity of from about 1 cps to about 2000 cps in combination with a virus inactivation agent; (B) a rheological agent providing for a composition viscosity of from about 1 cps to about 2000 cps in combination with a nasal secretion agent; (C) a virus inactivation agent in combination with a nasal secretion agent; and (D) a rheological agent providing for a composition viscosity of from about 1 cps to about 2000 cps, a virus inactivation agent, and a nasal secretion agent.

The present invention is also directed to a method of preventing and treating upper respiratory tract viral infections to result in encapsulation, inactivation, and removal of infectious respiratory viruses and/or viral strains, the method comprises administering a composition to the nasal cavity wherein the composition comprises; (a) a rheological agent providing for a composition viscosity of from about 1 cps to about 2000 cps; and (b) a buffer solution having a pH of from about 3.0 to about 5.5.

It has been found that the administration of select compositions to the nasal cavity can result in the encapsulation, inactivation, and/or removal of viruses and/or viral strains that can cause respiratory viral infections which are associated with the common cold and/or influenza. The methodologies defined herein provide for the administration of the compositions such that the viruses and/or viral strains are effectively treated using the procedure of encapsulation, activation, and removal, thereby resulting in highly effective methods of reducing and/or eliminating symptoms associated with the common cold and influenza.

### DETAILED DESCRIPTION OF THE INVENTION

The methods of the present invention provide for the encapsulation, inactivation, and/or removal of viruses and/or viral strains that are associated with the common cold and influenza. The methods involve administering compositions to the respiratory tract, especially the administration of compositions to the nasal cavity of the respiratory tract. These methods are highly effective in providing for the prevention and treatment of symptoms related to the common cold and influenza.

The term “encapsulation” as used herein refers to the envelopment of infectious viruses and/or viral strains within the matrix of the compositions defined herein, and the inhibition of the viruses and/or viral strains from making contact with cell receptors.

The term “inactivation” as used herein refers to the stoppage of virus particles' infectivity. In other words, “inactivation” means that the virus particles are no longer infectious. Inactivation materials defined herein can provide for temporary or permanent stoppage of virus particles infectivity, wherein temporary stoppage means that the inactivation material needs to be present for inactivation to occur and permanent stoppage mean that the inactivation material has provided for damage to virus particles such that the virus and/or viral strains cannot recover.

The term “secretion agent” as used herein refers to the physical removal of virus particles from the vicinity of their infection targets. Secretion agents defined herein stimulate a mild rhinorrhea such that virus particles and inflammatory mediators are washed away from the vicinity of cells that are susceptible to cold and/or influenza infections.

The term “respiratory tract” as used herein refers to the areas of the nose, mouth, tongue, and throat, including the mucosal membranes of the nose, mouth, tongue, and throat.

The compositions defined herein are administered to the respiratory tract to prevent and treat “cold and influenza-like symptoms”. As used herein “cold and influenza-like symptoms” refer to symptoms typically associated with respiratory tract viral infections. These symptoms include, but are not limited to, nasal congestion, chest congestion, sneezing, rhinorrhea, fatigue or malaise, coughing, fever, chills, body ache, sore throat, headache, and other known cold and influenza-like symptoms.

The terms “respiratory virus”, “respiratory viruses”, “viruses”, and “viral strains” are used interchangeably herein to refer to one or more viruses that are causal agents of cold and influenza-like symptoms. These viruses include Rhinovirus, Myxovirus (Influenza virus), Paramyxovirus (Parainfluenza virus), Respiratory Syncytial virus, Adenovirus and Coronavirus.

The method of the present invention includes the administration of compositions that can comprise, consist of, or consist essentially of the elements and limitations of the invention described herein, as well as any of the additional or optional ingredients, components, or limitations described herein.

All percentages, parts and ratios are by weight of the compositions, unless otherwise specified. All such weights as they pertain to listed ingredients are based on the specific ingredient level and, therefore, do not include carriers or by-products that may be included in commercially available materials, unless otherwise specified.

All documents cited herein, including publications, patent applications, and issued patents mentioned herein, are, in relevant part, incorporated herein by reference. Citation of any document is not an admission regarding any determination as to its availability as prior art to the present invention.

### Encapsulation Agent

The methods of the present invention include the administration of compositions that comprise an encapsulation agent that surrounds viruses and/or viral strains that are present in the respiratory tract area, and physically inhibits the viruses and/or viral strains from reaching target cell receptors of the respiratory tract. The encapsulation agent includes rheological agents that provide for the retention of the viruses and/or viral strains in areas of the respiratory tract such as the nasal cavity.

The rheological agent can be used in combination with a virus inactivation agent, or in combination with a nasal secretion agent, or the compositions can comprise a rheological agent, a virus inactivation agent, and a nasal secretion agent. Without being limited by theory, it is believed that the rheological agent provides for the retention of viruses and/or viral strains for further treatment by the virus inactivation agent and/or nasal secretion agent to maintain an environment hostile to viruses for improved prevention and treatment of cold and influenza-like symptoms. It has been found that the methods of the present invention are highly effective in the prevention and treatment of cold and influenza-like symptoms when the methods involve administering compositions that create an environment hostile to viruses. Such an environment can encapsulate, inactivate, and/or remove viruses in addition to providing for the deterrence of viruses further infecting respiratory tract areas, especially the nasal cavity.

The rheological agent can be included in the compositions of the present invention as an individual rheological agent or as a combination of rheological agents, provided that the total concentration of rheological agent ranges from about 0.01% to about 30%, preferably from about 0.1% to about 20%, more preferably from about 1% to about 15%, by weight of the composition.

The incorporation of the rheological agent into the compositions of the present invention typically results in a composition that has a viscosity in the range of from about 1 centipoise (cps) to about 2000 cps, preferably from about 1 cps to about 1000 cps, more preferably from about 5 cps to about 500 cps, most preferably from about 5 cps to about 300 cps. The viscosity of the compositions can be measured by any known or otherwise effective technique employed to determine viscosity. Generally, the viscosity of the compositions of the present invention is determined using known methods such as those described in ASTM D1824-87, ASTM D1084-88, and ASTM D2196-86. Typical viscometers employed to measure viscosity include the Brookfield Syncho-Lectric Viscometer and the Haake Viscometer. For example, when the Brookfield Syncho-Lectric Viscometer is utilized for viscosity measurements, this viscometer is typically equipped with a spindle 4 to measure viscosities of less than 8,000 centipoise at low shear rates at given rotational speeds. Likewise, when the Haake Viscometer is utilized, a suitable Haake Viscometer is the Rheostress 1 model that is equipped with a probe (i.e., spindle) such as probe C35/2T wherein the viscosity measurement is performed over a temperature range of 5° C. to 40° C. at 50 revolutions per minute (rpm)/second (sec).

Known rheological agents suitable for use herein are selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, and mixtures thereof.

Nonlimiting examples of suitable homopolymers of acrylic acid crosslinked with an allyl ether of pentaerythritol or an allyl ether of sucrose are available from B. F. Goodrich Company under the tradename “Carbopol”. Specific Carbopols include Carbopol 934, 940, 941, 956, 980, and mixtures thereof. Carbopol 980 is preferred among the carbopol rheological agents. Polymers of this type have slightly acidic carboxyl group substituents. Such polymers generally have a pH of around 3 in water and are generally used by neutralization during preparation of compositions to form viscous films and/or gels that can entrap viruses. When the compositions of the present invention comprise one or more Carpobol rheological agents, generally these polymers are used at concentrations ranging from about 0.01% to about 2.5% by weight of the composition.

Nonlimiting examples of suitable homopolymers of acrylic acid crosslinked with divinyl glycol are available from B. F. Goodrich Company as polycarbophils under the tradename “Noveon.”

Other nonlimiting examples of a rheological agent suitable for use herein include natural polymers, polymeric cellulose derivatives, polyvinyl pyrrolidones (PVPs), dextran polymers, polyethylene oxide polymers including Polyox-600, thermoreversible polymers, ionic responsive polymers, copolymers of polymethyl vinyl ether and maleic anhydride, and mixtures thereof. Polymeric cellulose derivatives and thermoreversible polymers are preferred.

Specific nonlimiting examples of natural polymers suitable for use as a rheological agent herein include arabic gums, tragacanth gums, agar polymers, xanthan gums, copolymers of alginic acid and sodium alginate, chitosan polymers, pectins, carageenans, pullulan polymers, modified starches, and mixtures thereof.

Specific nonlimiting examples of polymeric cellulose derivatives suitable for use as a preferred rheological agent herein include hydroxy alkyl cellulose polymers including hydroxypropylmethyl cellulose (HPMC) and hydroxypropyl cellulose (HPC), methyl cellulose polymers, carboxymethyl cellulose (CMC) polymers, salts of carboxymethyl cellulose including sodium salt of carboxymethyl cellulose, and mixtures thereof.

Specific nonlimiting examples of thermoreversible polymers suitable for use as a preferred rheological agent herein include poloxamers including those poloxamers sold under the Lutrol F-127 and Lutrol F-68 tradenames, ethylhydroxy ethylcellulose (EHEC), and mixtures thereof.

Specific nonlimiting examples of ionic responsive polymers suitable for use as a rheological agent herein include gelrite, gellan gum, Kelcogel F, and mixtures thereof.

Specific nonlimiting examples of copolymers of polymethyl vinyl ether and maleic anhydride suitable for use as a rheological agent herein include such copolymers sold under the Gantrez tradename including Gantrez S and Gantrez MS type copolymers.

The rheological agent suitable for use herein is more fully described in the Journal Pharmacy Pharmacology 53, pages 3-22, (2001 Edition); the International Journal of Pharmaceutics (1988, 1996 and 1998 Editions); and the Journal Controlled Release 62, pages 101-107, (1999 Edition); which descriptions are incorporated herein by reference.

### Inactivation Agent

The methods of the present invention include the administration of compositions that comprise a virus inactivation agent that provides for little or no infectivity of virus particles. The inactivation agent can temporarily or permanently prevent virus and/or viral strains infectivity to result in prevention and treatment of cold and influenza-like symptoms.

The compositions of the present invention can comprise one or more inactivation agents, provided that the total concentration of inactivation agent is from about 0.01% to about 20%, preferably from about 0.05% to about 10%, more preferably from about 0.10% to about 5%, by weight of the composition. The inactivation agent can be included in the composition in combination with the rheological agent and/or nasal secretion agent defined herein.

Inactivation agents suitable for use herein include metal compounds, surfactants, chelating agents, pyroglutamic acid, and mixtures thereof.

Nonlimiting examples of metal compounds suitable for use as an inactivation agent herein include those metal compounds commonly referred to as “metal salts” which comprise metal ion substituents selected from the group consisting of manganese (Mn), silver (Ag), zinc (Zn), tin (Sn), iron (Fe), copper (Cu), aluminum (Al), nickel (Ni), cobalt (Co), and mixtures thereof. Preferred metal compounds include those metal compounds which contain Cu, Fe, or Zn metal ions, or combinations thereof. Examples of such metal compounds include the metal compounds referred to as salicylates, fumarates, benzoates, glutarates, lactates, citrates, malonates, acetates, glycolates, thiosalicylates, adipates, succinates, gluconates, aspartates, glycinates, tartrates, malates, maleates, ascorbates, chlorides, sulphates, nitrates, phosphates, fluorides, iodides, pidolates, and mixtures thereof. The acetates, ascorbates, chlorides, benzoates, citrates, gluconates, glutarates, lactates, malates, malonates, salicylates, succinates, sulphates, and mixtures thereof are preferred metal compounds.

Specific examples of a metal compound suitable for use herein include zinc acetate, zinc chloride, zinc ascorbate, zinc gluconate, zinc pidolate, zinc succinate, zinc sulphate, zinc chloride, and mixtures thereof. Zinc acetate is the most preferred metal compound.

When the compositions of the present invention comprise a metal compound containing zinc ion, it is believed that the zinc ion provides for antiviral properties that results in the inactivation of viruses and/or viral strains. Furthermore, it is known that metal ions such as iron, silver, copper, and zinc can provide antiviral properties for the prevention and treatment of cold and influenza-like symptoms. Particularly, zinc and its possible effects on common colds has been extensively documented, The Handbook for Curing the Common Cold, George A. Eby, published 1994, George Eby Research, Texas, USA. The mechanism of its action is thought to be multifactorial. Zinc ions have been shown to be both antiviral and antibacterial. They are believed to inhibit cleavage of rhinovirus polypeptides, preventing replication and formation of infective virions. Zinc ions reduce the ability of rhinoviruses to penetrate cell membranes, partly by lowering expression of intercellular adhesion molecule ICAM. Zinc ions have also been shown to stimulate T-cell lyphocytes, including production of the natural antiviral, interferon-gamma. They stabilize cell plasma membranes, protecting cells from cytotoxic agents, and preventing cell leakage.

Nonlimiting examples of surfactants suitable for use as an inactivation agent herein include nonionic surfactants, anionic surfactants, cationic surfactants, amphoteric surfactants, zwtterionic surfactants, and mixtures thereof. Nonionic and anionic surfactants are preferred.

Specific nonlimiting examples of nonionic surfactants include amine oxides such as N,N-dimethyldodecylamine-N-oxide, available from Procter & Gamble Chemical, USA; Nonoxynol-9, available from Shanghai Longsheng Corporation, China; Span, available from Dewolf chemical Inc. East Province, R102914; The Brij class of surfactants, such as Brij 76 (Steareth-10) and Brij 56 (Ceteth-10), available from Sigma-Aldrich; Sorbitan esters known as Tweens, eg Tween 80, available from Sigma-Aldrich; and mixtures thereof.

Specific nonlimiting examples of anionic surfactants include alkyl lauryl sulphate and alkyl ether sulphate or their sodium salts, available from Surfachem Limited, Leeds, UK; ammonium lauryl sulphate, known as Genapol LSA, available from Clariant Limited, Leeds, UK; Sodium C14-C17 alkyl sulphonate, known as Hostapur, available from Clariant Limited, Leeds, UK; and mixtures thereof.

Nonlimiting examples of chelating agents suitable for use as an inactivation agent herein include phytic acid; alkaline salts of ethylene diamine tetraacetic acid (EDTA) including disodium, calcium, and zinc salts of EDTA; tetrasodium EDTA; sodium hexametaphosphate (SHMP); di(hydroxyethyl)glycine; 8-hydroxyquinoline; and mixtures thereof.

Nonlimiting example of a pyroglutamic acid suitable for use as an inactivation agent herein includes those pyroglutamic acid compounds collectively referred to as stereoisomers and tautomers of pyroglutamic acid. Pyroglutamic acid, which is also referred to as pyrrolidone carboxylic acid has two stereoisomers (D and L) and each are preferred for use herein. Pharmaceutically acceptable salts of pyroglutamic acid are also suitable for use herein.

The D stereoisomer of pyroglutamic acid is also known by the following names: D-Proline, 5-oxo-(+)-2-Pyrrolidone-5-carboxylic acid, (+)-Pyroglutamic acid, (R)-2-Pyrrolidone-5-carboxylic acid, 5-Oxo-D-proline, D-2-Pyrrolidone-5-carboxylic acid, D-Pyroglutamic acid, D-Pyrrolidinonecarboxylic acid, and D-Pyrrolidonecarboxylic acid.

The L stereoisomer of pyroglutamic acid is also known by the following names: L-Proline, 5-oxo-(−)-2-Pyrrolidone-5-carboxylic acid, (−)-Pyroglutamic acid, (5S)-2-Oxopyrrolidine-5-carboxylic acid, (S)-(−)-2-Pyrrolidone-5-carboxylic acid, (S)-2-Pyrrolidone-5-carboxylic acid, (S)-5-Oxo-2-pyrrolidinecarboxylic acid, (S)-Pyroglutamic acid, 2-L-Pyrrolidone-5-carboxylic acid, 2-Pyrrolidinone-5-carboxylic acid, 5-Carboxy-2-pyrrolidinone, 5-Oxo-L-proline, 5-Oxoproline, 5-Pyrrolidinone-2-carboxylic acid, Glutimic acid, Glutiminic acid, L-2-Pyrrolidone-5-carboxylic acid, L-5-Carboxy-2-pyrrolidinone, L-5-Oxo-2-pyrrolidinecarboxylic acid, L-5-Oxoproline, L-Glutamic acid, gamma-lactam, L-Glutimic acid, L-Glutiminic acid, L-Pyroglutamic acid, L-Pyrrolidinonecarboxylic acid, L-Pyrrolidonecarboxylic acid, Oxoproline, PCA, Pidolic acid, Pyroglutamic acid, Pyrrolidinonecarboxylic acid, Pyrrolidone-5-carboxylic acid, and Pyrrolidonecarboxylic acid.

The DL form of pyroglutamic acid (a mixture of the D and L stereoisomers) is known by the following names: DL-Proline, 5-oxo-(.+−.)-2-Pyrrolidone-5-carboxylic acid, (.+−.)-Pyroglutamic acid, 5-Oxo-DL-proline, DL-2-Pyrrolidinone-5-carboxylic acid, DL-2-Pyrrolidone-5-carboxylic acid, DL-Pyroglutamate, DL-Pyroglutamic acid, DL-Pyrrolidonecarboxylic acid, and Oxoproline. The DL form is also commercially available from Ajinomoto under the tradenames Ajidew A 100 and Ajidew N 50 (Na-PCA).

Some of the above-listed stereoisomers are commercially available from UCIB, France via Barnet Products Corp., New Jersey. Such compounds are sold under trade names like Cuivridone (Cu-PCA) and L-FER Pidolate (Fe-PCA), and Pidolidone.

When the compositions of the present invention comprise pyroglutamic acid in combination with an organic acid secretion agent having a pKa value from about 3.0 to about 5.5, it has been shown that this combination provides for a surface pH of the nasal cavity tissue of from about pH 3.0 to 5.5.

### Nasal Secretion Agent

The methods of the present invention include the administration of compositions that comprise a nasal secretion agent that provides for the removal of viruses and/or viral strains from the respiratory tract area, especially from the nasal cavity. The nasal secretion agent stimulates a mild rhinorrhea such that virus particles and inflammatory mediators are washed away from affected cell receptors located in respiratory tract areas such as the nasal cavity.

The compositions of the present invention can comprise one or more nasal secretion agents, provided that the total concentration of nasal secretion agent is from about 0.001% to about 10%, preferably from about 0.005% to about 5%, more preferably from about 0.01% to about 1%, by weight of the composition. The nasal secretion agent can be included in the composition in combination with the rheological agent and/or inactivation agent defined herein.

Nasal secretion agents suitable for use herein include organic acids, aromatic plant extracts, hypertonic solutions, and mixtures thereof.

Nonlimiting examples of organic acids suitable for use herein as a nasal secretion agent include ascorbic acid, monocarboxylic acids, dicarboxylic acids, tricarboxylic acids, and mixtures thereof.

Specific nonlimiting examples of suitable monocarboxylic, dicarboxylic, or tricarboxylic acids include salicylic, fumaric, benzoic, glutaric, lactic, citric, malonic, acetic, glycolic, malic, adipic, succinic, aspartic, phthalic, tartaric, glutamic, gluconic, and mixtures thereof.

Nonlimiting examples of aromatic plant extracts suitable for use as a nasal secretion agent herein include pepper extracts, garlic extracts, onion extracts, mustard extracts, and mixtures therof. Specific nonlimiting examples of suitable pepper extracts include capsaicin, capsicum, and mixtures thereof.

Nonlimiting examples of hypertonic solutions suitable for use as a nasal secretion agent herein include sodium chloride at concentrations with an osmolarity of from about 280 milliosmoles to about 450 milliosmoles, and mixtures thereof.

### Buffer Solution

The methods of the present invention include the administration of compositions that comprise an encapsulation agent in combination with a buffer solution having a pH of from about 3.0 to about 5.5. Combinations of an encapsulation agent and a buffer solution have also been found to provide for compositions that are effective in the encapsulation, inactivation, and removal of infectious respiratory viruses and/or viral strains to result in the prevention and treatment of respiratory tract viral infections.

Nonlimiting examples of buffering agents which provide for buffer solutions suitable for use herein include fumarates, benzoates, lactates, citrates, succinates, tartrates, chlorides, sulphates, phosphates, and mixtures thereof.

### Pharmaceutically Acceptable Vehicle

The methods of the present invention include the administration of compositions to respiratory tract areas, particularly the nasal cavity. The compositions are typically administered to the respiratory tract areas as formulations comprising a pharmaceutically acceptable vehicle or carrier system. Any pharmaceutically acceptable vehicle in the form of a liquid, solid, or gas is suitable for the delivery of the respiratory tract compositions to prevent and treat cold and influenza-like symptoms.

The compositions of the present invention can be administered in product forms such as droppers, pump sprayers, pressurized sprayers, atomizers, air inhalation devices and the like. Depending on the desired form and delivery device to be used, the compositions of the present invention can be combined with pharmaceutically acceptable vehicles such as water, water-miscible solvents including ethanol, propylene glycol, polyethylene glycol, transcutol, glycerol, and other known or otherwise effective water-miscible solvents; liquid aerosol propellants; and mixtures thereof. Preferably these vehicles are isotonic with human plasma.

When the compositions are administered using water as a pharmaceutically acceptable vehicle, the water is preferably purified or de-ionized water and is free of organic impurities. The concentration of water utilized to formulate the compositions into a final product form for delivery to respiratory tract areas ranges from about 40% to about 99.98%, preferably from about 80% to about 99.95%, by weight of the final product formulation.

When the compositions of the present invention are administered using a solid pharmaceutically acceptable vehicle, the vehicle may be applied in a powder form. In other words, the compositions of the present invention can be applied as a solid powder containing the essential ingredients and any optional components described herein with or without any known or otherwise effective solidification aids. However, pharmaceutically acceptable solid vehicles can be added to provide aid in processing of the compositions, to aid in the consistency of the compositions, to provide for improved stability, to facilitate handling, for hygroscopicity benefits, and so forth. Pharmaceutically acceptable solid vehicle materials include ingredients such as particulate and powder fillers, for example, a lactose powder. For respiratory tract compositions in the form of nasal compositions that are administered using a solid powder pharmaceutically acceptable vehicle, the particle size of the powder is typically greater than 10 microns, especially when the nasal composition is a nasal inhalant.

### Optional Components

The compositions of the present invention may further comprise one or more optional components known or otherwise effective for use in pharmaceutical compositions, provided that the optional components are physically and chemically compatible with the essential components described hereinabove, or do not otherwise unduly impair product stability, aesthetics, or performance. Optional components suitable for use herein include materials such as pH adjusting agents, preservatives, sensates, sweeteners, flavors, volatile oils, mucilages, and so forth. The optional components can be included in the compositions at concentrations ranging from about 0.001% to about 20%, preferably from about 0.01% to about 10%, by weight of the composition.

The compositions of the present invention can optionally comprise homeopathic ingredients. A detailed, but not necessarily a complete list, of such homeopathic ingredients is found in The Homeopathic Pharmacopoeia of the United States, 1999 ed., published by The Pharmacopoeia Convention of the American Institute of Homeopathy, ©1982, Vol. 1-4, which descriptions are incorporated herein by reference. Specific nonlimiting examples of known, homeopathic, or otherwise effective, optional components suitable for use herein are described in more detail hereinbelow.

A specific nonlimiting example of an optional component suitable for use herein include optional pH adjusting agents. Optional pH adjusting agents can be included in the compositions of the present invention to adjust the pH of the compositions to values less than about 4.5. Therefore, when the compositions are applied to respiratory tract areas such as nasal tissues, the pH of the composition on the nasal tissues remains from about 3.0 to about 5.5, but is not so low as to cause irritation of the nasal tissues. Such optional pH adjusting agents include those normally associated with use in nasal compositions including compounds such as sodium bicarbonate, sodium phosphate, sodium hydroxide, ammonium hydroxide, sodium stannate, triethanolamine, sodium citrate, disodium succinate, and mixtures thereof. If present, the optional pH adjusting agents are generally included at concentrations ranging from about 0.01% to about 5.0% by weight of the composition.

Another specific nonlimiting example of an optional component suitable for use herein include optional preservatives. Preservatives can optionally be included to prevent microbial contamination that can be attributed to dosing devices or the application of the composition to the nose. Such optional preservatives include those normally associated with use in nasal compositions including benzalkonium chloride, chlorhexidine gluconate, phenyl ethyl alcohol, phenoxyethanol, benzyl alcohol, sorbic acid, thimerosal, phenylmercuric acetate, and mixtures thereof.

### Method of Manufacture

The compositions of the present invention may be prepared by any known or otherwise effective technique suitable for providing a pharmaceutical composition that provides a therapeutic benefit in the prevention and treatment of cold and influenza-like symptoms. The methods of the present invention include the administration of compositions to the respiratory tract, wherein these compositions are manufactured into final product forms of liquids, sprays, powders, inhalants, pumps, drops, and so forth for administration to the respiratory tract areas to prevent and treat symptoms due to respiratory tract viral infections.

When the compositions are administered using a pharmaceutically acceptable vehicle such as a liquid to deliver the compositions in product forms of sprays, pumps, droplets, and the like, the compositions are generally prepared by solubilizing a rheological agent in a liquid vehicle such as water. While stirring, a virus inactivation agent and/or nasal secretion agent are then added to the rheological agent solution. Next, a sensate mix is added while the solution is allowed to continue stirring. The sensate mix is typically added as a premix solution that can contain a combination of ingredients such as a combination of ethanol, menthol, peppermint oil, and spearmint oil. The pH of the resultant product should be between about 3.0 and about 5.5, however, a pH adjusting agent such as sodium hydroxide and/or disodium succinate can be added to maintain the pH of the resultant product to values less than about 4.5. These compositions are administered as respiratory tract compositions in their liquid final product forms, wherein the liquid is suitable for incorporation into fill dropper vials for spraying into respiratory tract areas such as the nostrils or turbinates to result in effective prevention and treatment of cold and influenza-like symptoms. Typically, from about 1 microliter (μl) to about 500 microliters (μls) of the composition are sprayed into each nostril or turbinate.

When the compositions of the present invention are administered using a pharmaceutically acceptable vehicle such as a powder, the compositions are generally prepared by dry blending a rheological agent, and/or virus inactivation agent, and/or nasal secretion agent using a V-mixer. A pH adjusting agent such as sodium citrate can be added to the dry blend. The dry blend is then micronized using a fluid energy mill. The resultant micronized dry blend is then dry mixed with a powder filler such as lactose powder. This final powder respiratory tract composition can optionally be coated with a sensate premix using known spray coating techniques. The final powder respiratory tract composition can be filled into a nasal inhalation metering pump to prevent and treat symptoms of the cold and influenza, wherein about 10 milligrams (mgs) of the final powder can be administered to a respiratory tract area such as a nostril or a turbinate.

As stated herein, the compositions of the present invention are suitable for administration to the respiratory tract in final product forms of liquids, sprays, pumps, inhalants, powders, and so forth. Suitable devices utilized in the administration of these final respiratory tract compositions include those commonly employed or otherwise effective liquid containers, droppers, spray containers including pressurized sprayers, pump containers, inhalation devices, powder containers, atomizers, and so forth.

### Method of Treatment

The present invention is directed to methods of preventing and treating respiratory tract viral infections by administering compositions described herein to respiratory tract areas such as the nasal cavity. Generally, a safe and effective amount of the compositions is applied to the respiratory tract area, particularly the nasal cavity. In this context, the term “safe and effective amount” refers to an amount which provides a therapeutic benefit with minimal or no adverse reactions.

As referred to herein, the methods of preventing and treating respiratory tract viral infections include any known or otherwise effective method of preventing and treating viruses and/or viral strains that can affect the respiratory tract to result in symptoms associated with the common cold and influenza.

To prevent and treat respiratory tract viral infections, a safe and effective amount of the compositions of the present invention are administered to the respiratory tract. The safe and effective amount will depend on factors such as the type of composition administered, for example, the compositions of the present invention can be administered using product forms such as liquids, sprays, powders, inhalants, pumps, drops, and the like.

A preferred method of treating and preventing respiratory tract viral infections involves spraying the compositions of the present invention into the nasal cavity. For respiratory tract compositions in the form of nasal sprays, effective amounts of from about 1 microliter to about 500 microliters, preferably from about 1 microliter to about 150 microliters, are sprayed into each nostril or turbinate of the nasal cavity one or more times to administer an effective method of preventing and treating respiratory tract viral infections. Typically, about 50 microliters of the nasal spray is administered two to three times into each nostril or turbinate as an effective method of preventing and treating respiratory tract viral infections. For respiratory tract compositions in the form of diluted nasal sprays or nasal irrigations, from about 0.1 milliliters (mls) to about 50 milliliters are sprayed into each nostril or turbinate one or more times. It has been found that upon spraying the compositions into the nasal cavity, the infectious viruses and/or viral strains are encapsulated, inactivated, and/or removed from the nasal cavity to alleviate cold and influenza-like symptoms that can be contributed to the viruses and/or viral strains.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Exemplary respiratory tract compositions of the present invention are exemplified in Table II hereinbelow. These respiratory tract compositions preferably comprise a sensate premix exemplified in Table I hereinbelow. The exemplified sensate premixes of Table I provide for respiratory tract compositions that are aesthetically pleasing in taste, flavor, coolness, smell, and the like.

The respiratory tract compositions exemplified hereinbelow in Table II are suitable for spraying into respiratory tract areas such as the nostrils or turbinates for effective prevention and treatment of cold and influenza-like symptoms. Typically, from about 1 microliter to about 500 microliters of the composition are sprayed into each nostril or turbinate.

TABLE I

| | Sensate Mix A | Sensate Mix B |
|---|---|---|
| Component | (Wt. %) | (Wt. %) |
| Ethanol | 47.16 | — |
| Menthol | 29.41 | 11.565 |
| Peppermint Oil | 17.61 | — |
| Spearmint Oil | 5.82 | — |
| Phenyl Ethyl Alcohol | — | 77.495 |
| Camphor | — | 6.971 |
| Eucalyptol | — | 3.969 |
| Total: | 100 | 100 |

TABLE II

| | Sample 1 | Sample 2 | Sample 3 | Sample 4 | Sample 5 |
|---|---|---|---|---|---|
| Component | (Wt. %) | (Wt. %) | (Wt. %) | (Wt. %) | (Wt. %) |
| Hydroxypropylmethyl cellulose | 1.00 | — | 1.00 | 1.00 | 1.00 |
| Rheological Agent¹ | | | | | |
| Lutrol F-127 Rheological Agent² | — | 15.0 | — | — | — |
| Zinc Acetate Virus Inactivation Agent³ | 0.12 | 0.12 | — | — | 0.12 |
| Amine oxide Virus Inactivation Agent⁴ | — | — | 0.10 | — | — |
| Nonoxynol-9 Virus Inactivation Agent⁵ | — | — | — | 0.10 | — |
| Succinic Acid Nasal Secretion Agent⁶ | 1.00 | 1.00 | — | — | — |
| Acetic Acid Nasal Secretion Agent⁷ | — | — | 0.05 | — | — |
| Capsaicin Nasal Secretion Agent⁸ | — | — | — | 0.01 | — |
| Sodium Chloride Nasal Secretion Agent⁹ | — | — | — | — | 2.00 |
| Polysorbate 80 | 0.05 | 0.05 | 0.05 | 0.05 | 0.05 |
| Sodiun Saccharin | 0.025 | 0.025 | 0.025 | 0.025 | 0.025 |
| Phenyl ethyl alcohol | 0.037 | 0.037 | 0.037 | 0.037 | 0.037 |
| Sensate Mix A | 0.067 | — | — | — | — |
| Sensate Mix B | — | 0.039 | — | — | — |
| Disodium succinate | 1.00 | 0.50 | — | — | — |
| Deionized Water | q.s. 100 | q.s. 100 | q.s. 100 | q.s. 100 | q.s. 100 |

Wt. % - weight percent
¹Hydroxypropylmethyl cellulose available from Colorcon Ltd, Kent, UK
²Lutrol F-127 available from BASF Speciality Chemicals, Mount Oliver, NJ, USA
³Zinc acetate dihydrate available from Verdugt B. V., Belgium
⁴Amine oxide available from Procter & Gamble Chemicals USA
⁵Nonoxynol-9 available from Shanghai Langsheng Corporation
⁶Succinic acid available from DSM Fine Chemicals, UK.
⁷Acetic acid available from Post Apple Scientific, PA, USA
⁸Capsaicin available from Steve Weiss & Co, New York, USA
⁹Sodium chloride available from Alfa AESAR, MA, USA

While particular embodiments suitable for use in the method of the present invention have been described, it will be obvious to those skilled in the art that various changes and modifications of the present invention can be made without departing from the spirit and scope of the invention. It is intended to cover, in the appended claims, all such modifications that are within the scope of this invention.

1. A method of preventing and treating upper respiratory tract viral infections by administering a composition to the nasal cavity, wherein the composition comprises:
(a) from about 0.01% to about 30% by weight of a rheological agent; and
(b) from about 0.01% to about 20% by weight of a virus inactivation agent;
wherein the composition has a viscosity of from about 1 cps to about 2000 cps.

2. The method of claim 1 wherein the composition has a viscosity of from about 5 cps to about 500 cps.

3. The method of claim 1 wherein the rheological agent is selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric cellulose derivatives, polyvinyl pyrrolidones (PVPs), dextran polymers, polyethylene oxide polymers, thermoreversible polymers, ionic responsive polymers, copolymers of polymethyl vinyl ether and maleic anhydride, and mixtures thereof.

4. The method of claim 3 wherein the rheological agent is a cellulose derivative selected from the group consisting of hydroxypropylmethyl celluloses, hydroxypropyl celluloses, methyl cellulose polymers, carboxymethyl cellulose polymers, salts of carboxymethyl cellulose, and mixtures thereof.

5. The method of claim 3 wherein the rheological agent is a thermoreversible polymer selected from the group consisting of poloxamers, ethylhydroxy ethylcelluloses, and mixtures thereof.

6. The method of claim 1 wherein the virus inactivation agent is selected from the group consisting of a metal compound, a surfactant, a chelating agent, pyroglutamic acid, and mixtures thereof.

7. The method of claim 6 wherein the metal compound is selected from the group consisting of salicylates, fumarates, benzoates, glutarates, lactates, citrates, malonates, acetates, glycolates, thiosalicylates, adipates, succinates, gluconates, aspartates, glycinates, tartrates, malates, maleates, ascorbates, chlorides, sulphates, nitrates, phosphates, fluorides, iodides, pidolates, and mixtures thereof.

8. The method of claim 7 wherein the metal compound is an acetate metal compound.

9. The method of claim 8 wherein the acetate is zinc acetate.

10. The method of claim 6 wherein the surfactant is selected from the group consisting of nonionic surfactants, anionic surfactants, cationic surfactants, amphoteric surfactants, zwtterionic surfactants, and mixtures thereof.

11. The method of claim 6 wherein the chelating agent is selected from the group consisting of phytic acid, disodium salts of ethylene diamine tetraacetic acid (EDTA), calcium salts of EDTA, zinc salts of EDTA, tetrasodium EDTA, sodium hexametaphosphate (SHMP), di(hydroxyethyl)glycine, 8-hydroxyquinoline, and mixtures thereof.

12. The method of claim 1 wherein the composition further comprises from about 0.001% to about 10% by weight of a nasal secretion agent selected from the group consisting of an organic acid, an aromatic plant extract, a hypertonic solution, and mixtures thereof.

13. The method of claim 12 wherein the organic acid is selected from the group consisting of ascorbic acid, salicylic acid, fumaric acid, benzoic acid, glutaric acid, lactic acid, citric acid, malonic acid, acetic acid, glycolic acid, malic acid, adipic acid, succinic acid, aspartic acid, phthalic acid, tartaric acid, glutamic acid, gluconic acid, and mixtures thereof.

14. The method of claim 12 wherein the aromatic plant extract is selected from the group consisting of pepper extracts, garlic extracts, onion extracts, mustard extracts, and mixtures thereof.

15. The method of claim 12 wherein the hypertonic solution is selected from the group consisting of sodium chloride at concentrations with an osmolarity of from about 280 milliosmoles to about 450 milliosmoles, and mixtures thereof.

16. The method of claim 13 wherein the composition has a pH in the range of from about 3.0 to about 5.5.

17. The method of claim 16 wherein the composition further comprises a pH adjusting agent selected from the group consisting of sodium bicarbonate, sodium phosphate, sodium hydroxide, ammonium hydroxide, sodium stannate, triethanolamine, sodium citrate, disodium succinate, and mixtures thereof.

18. The method of claim 1 wherein the composition is a nasal spray comprising from about 40% to about 99.98% by weight of a pharmaceutically acceptable vehicle selected from the group consisting of water, ethanol, propylene glycol, polyethylene glycol, transcutol, glycerol, a liquid aerosol propellant, and mixtures thereof.

19. A method of preventing and treating upper respiratory tract viral infections to result in encapsulation, inactivation, and removal of the viral infections, the method comprises administering a composition to the nasal cavity wherein the composition comprises:
(a) a rheological agent; and
(b) a buffer solution having a pH of from about 3.0 to about 5.5;
wherein the composition has a viscosity of from about 1 cps to about 2000 cps.

20. The method of claim 19 wherein the rheological agent is selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric cellulose derivatives, polyvinyl pyrrolidones (PVPs), dextran polymers, polyethylene oxide polymers, thermoreversible polymers, ionic responsive polymers, copolymers of polymethyl vinyl ether and maleic anhydride, and mixtures thereof.

21. The method of claim 20 wherein the rheological agent is a cellulose derivative selected from the group consisting of hydroxypropylmethyl celluloses, hydroxypropyl celluloses, methyl cellulose polymers, carboxymethyl cellulose polymers, salts of carboxymethyl cellulose, and mixtures thereof.

22. The method of claim 20 wherein the rheological agent is a thermoreversible polymer selected from the group consisting of poloxamers, ethylhydroxy ethylcelluloses, and mixtures thereof.

23. The method of claim 19 wherein the buffer solution comprises a buffering agent selected from the group consisting of fumarates, benzoates, lactates, citrates, succinates, tartrates, chlorides, sulphates, phosphates, and mixtures thereof.

24. A method of preventing and treating upper respiratory tract viral infections comprising the steps of:
(a) encapsulating one or more respiratory viruses; and
(b) inactivating the respiratory viruses.

25. The method of claim 24 wherein the encapsulating step comprises the administration of a composition comprising a rheological agent selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric cellulose derivatives, polyvinyl pyrrolidones (PVPs), dextran polymers, polyethylene oxide polymers, thermoreversible polymers, ionic responsive polymers, copolymers of polymethyl vinyl ether and maleic anhydride, and mixtures thereof.

26. The method of claim 25 wherein the composition has a viscosity of from about 1 cps to about 2000 cps.

27. The method of claim 26 wherein the composition comprises from about 0.01% to about 30% by weight of the rheological agent.

28. The method of claim 24 wherein the inactivating step comprises the administration of a composition comprising a virus inactivation agent selected from the group consisting of a metal compound, a surfactant, a chelating agent, pyroglutamic acid, and mixtures thereof.

28. The method of claim 28 wherein the composition comprises from about 0.01% to about 20% by weight of the virus inactivation agent.

29. The method of claim 24 wherein the method further comprises the step of:
(c) removing the respiratory viruses from the respiratory tract.

30. The method of claim 29 wherein the removing step comprises the administration of a composition comprising a nasal secretion agent selected from the group consisting of an organic acid, an aromatic plant extract, a hypertonic solution, and mixtures thereof.

31. The method of claim 30 wherein the composition comprises from about 0.001% to about 10% by weight of the nasal secretion agent.

32. The method of claim 31 wherein the composition has a pH in the range of from about 3.0 to about 5.5.

33. The method of claim 32 wherein the composition is a nasal spray comprising from about 40% to about 99.98% by weight of a pharmaceutically acceptable vehicle selected from the group consisting of water, ethanol, propylene glycol, polyethylene glycol, transcutol, glycerol, a liquid aerosol propellant, and mixtures thereof.
